(19) United States
(12) Patent Application Publication
(10) Pub. No.: US 2019/0083163 A1
(43) Pub. Date: Mar. 21, 2019

(54) ELECTROSURGICAL LOOP INSTRUMENTS FOR RESECTING TISSUE

(71) Applicant: COVIDIEN LP, Mansfield, MA (US)

(72) Inventors: SCOTT J. PRIOR, SHELTON, CT (US); NIKOLAI D. BEGG, WAYLAND, MA (US); PAUL D. DiCesare, EASTON, CT (US)

(21) Appl. No.: 16/103,358

(22) Filed: Aug. 14, 2018

Related U.S. Application Data
(60) Provisional application No. 62/561,241, filed on Sep. 21, 2017.

(51) Int. Cl. A61B 18/14; A61B 18/12

(52) U.S. Cl. CPC A61B 18/14; A61B 2018/00601; A61B 18/1233

(57) ABSTRACT

An electrosurgical instrument is provided including a handle assembly having an actuator and a shaft extending distally from the handle assembly. An electrosurgical loop is adapted to connect to a source of energy and is operably supported at a distal end portion of the shaft. The electrosurgical loop is movable relative to the shaft between a first retracted state and a first expanded state. A support structure is operably supported at the distal end portion of the shaft and is disposed about the electrosurgical loop. The support structure is movable relative to the shaft between a second retracted state and a second expanded state. Actuation of the actuator moves at least one of the electrosurgical loop or the support structure from the retracted state to the expanded state. The electrosurgical loop and the support structure are movable relative to each other.

### CROSS REFERENCE TO RELATED APPLICATION

The present application claims the benefit of and priority to U.S. Provisional Application Ser. No. 62/561,241, filed on Sep. 21, 2017 the entire contents of which are incorporated herein by reference.

### TECHNICAL FIELD

The present disclosure relates to electrosurgical instruments, and more particularly, to electrosurgical loop instruments for resecting tissue.

### BACKGROUND

Electrosurgical instruments and techniques are widely used in surgical procedures because they generally reduce patient bleeding and trauma associated with cutting operations. One such electrosurgical instrument is an electrosurgical loop instrument. Generally, electrosurgical loop instruments include a handle, a shaft extending from the handle, and an electrosurgical loop disposed at a distal end portion of the shaft. The electrosurgical loop may be extended and retracted to ensnare tissue to resect tissue. The resection may be performed within a specimen bag or, alternatively, the resected tissue may be placed into a specimen bag after resection, to keep the resected tissue away from healthy tissue.

However, electrosurgical loops can lack structural rigidity and therefore limit user control over their placement around tissue. Moreover, external surfaces (e.g., specimen bags) risk exposure to electrical energy and/or heat from the electrosurgical loop during the course of a procedure, even when the electrosurgical loop is not receiving electrical energy.

### SUMMARY

Accordingly, a need exists for an electrosurgical loop that provides for improved user control and that can protect external surfaces from exposure to electrical energy and/or heat from electrosurgical loops during the course of a surgical procedure.

According to an aspect of the present disclosure, an electrosurgical instrument is provided including a handle assembly having an actuator and a shaft extending distally from the handle assembly. An electrosurgical loop is adapted to connect to a source of energy and is operably supported at a distal end portion of the shaft. The electrosurgical loop is movable relative to the shaft between a first retracted state and a first expanded state. A support structure is operably supported at the distal end portion of the shaft and is disposed about the electrosurgical loop. The support structure is movable relative to the shaft between a second retracted state and a second expanded state. Actuation of the actuator moves at least one of the electrosurgical loop or the support structure from the retracted state to the expanded state. The electrosurgical loop and the support structure are movable relative to each other.

In embodiments, the support structure includes an inner surface and an outer surface. The inner surface is configured for contact with the electrosurgical loop and the outer surface is configured for contact with an external surface. The support structure is configured to shield the external surface from heat and energy from the electrosurgical loop.

In some embodiments, the support structure is a band disposed about an outer periphery of the electrosurgical loop. The band defines a width that is greater than a width of the electrosurgical loop.

In certain embodiments, the electrosurgical loop includes a metal selected from the group consisting of copper, copper alloy, stainless steel, tungsten, platinum, niobium, and molybdenum.

In embodiments, the actuator is a movable trigger movable relative to a fixed handle of the handle assembly.

In some embodiments, a second actuator is coupled to the handle assembly and is configured to move the electrosurgical loop relative to the shaft and is configured to move the support structure relative to the shaft.

In certain embodiments, the electrosurgical loop is connected to a source of electrical energy to energize the electrosurgical loop for resection of tissue therewith.

In embodiments, the electrosurgical loop includes a sleeve surrounding a portion of the electrosurgical loop.

In some embodiments, the support structure is configured to provide structural reinforcement to the electrosurgical loop.

In certain embodiments, the support structure includes an insulative material.

According to another aspect of the present disclosure, an electrosurgical instrument is provided, including a handle assembly having at least one actuator and a shaft extending distally from the handle assembly. An electrosurgical loop is operably supported at a distal end portion of the shaft. The electrosurgical loop includes first and second arms pivotably connected to each other at distal end portions thereof. The first and second arms are pivotable relative to one another to move the electrosurgical loop between a retracted state and an expanded state. A first stiffening sleeve is disposed partially about the first arm and a second stiffening sleeve is disposed partially about the second arm. The first and second stiffening sleeves are configured to provide structural reinforcement to the electrosurgical loop.

In embodiments, a pivot pin pivotably connects the first and second arms to each other.

In some embodiments, the pivot pin is formed from an electrically conductive material configured to permit electrical energy transmission between the first and second arms.

In certain embodiments, the first and second stiffening sleeves are formed from a spring steel configured to enable the electrosurgical loop to move between a non-deflected position and a deflected position.

In embodiments, a band is disposed about an outer periphery of the electrosurgical loop and defines a width that is greater than a width of the electrosurgical loop.

In some embodiments, the band includes an inner surface and an outer surface. The inner surface is configured for contact with the electrosurgical loop and the outer surface is configured for contact with an external surface. The band is configured to shield the external surface from heat and energy from the electrosurgical loop.

In certain embodiments, the at least one actuator includes a first trigger and a second trigger. The first trigger is configured to move the electrosurgical loop and the second actuator is configured to move the band about the electrosurgical loop. The electrosurgical loop and the band are movable relative to each other.

In embodiments, the electrosurgical loop is connected to a source of electrical energy to energize the electrosurgical loop for resection of tissue therewith.

In some embodiments, the electrosurgical loop includes a metal selected from the group consisting of copper, copper alloy, stainless steel, tungsten, platinum, niobium, and molybdenum.

### BRIEF DESCRIPTION OF THE DRAWINGS

Objects and features of the present disclosure will become apparent to those of ordinary skill in the art when descriptions thereof are read with reference to the accompanying drawings, of which:

FIG. 1 is a side view of an electrosurgical instrument in accordance with the present disclosure;

FIG. 2 is perspective view of a distal end portion of the electrosurgical instrument of FIG. 1 including an electrosurgical loop and a support structure disposed about the electrosurgical loop.

FIG. 3A is a side view of the distal end portion of the electrosurgical instrument of FIG. 1 with the electrosurgical loop and the support structure in an extended state;

FIG. 3B is a side view of the distal end portion of the electrosurgical instrument of FIG. 1 with the electrosurgical loop and the support structure in a first retracted state;

FIG. 3C is a side view of the distal end portion of the electrosurgical instrument of FIG. 1 with the electrosurgical loop and the support structure in a second retracted state;

FIG. 4A is a side view of the distal end portion of the electrosurgical instrument of FIG. 1 with the electrosurgical loop in the first retracted state and the support structure in the extended state;

FIG. 4B is a side view of the distal end portion of the electrosurgical instrument of FIG. 1 with the electrosurgical loop in the first retracted state and the support structure in the first retracted state;

FIG. 4C is a side view of the distal end portion of the electrosurgical instrument of FIG. 1 with the electrosurgical loop in the second retracted state and the support structure in the extended state;

FIG. 5 is a schematic diagram of the distal end portion of the electrosurgical instrument of FIG. 1 in use resecting tissue within a specimen bag of a specimen retrieval device disposed within an internal body cavity;

FIG. 6A is a side view of a distal end portion of another electrosurgical instrument in accordance with the present disclosure; and

FIG. 6B is a side view of the distal end portion of the electrosurgical instrument of FIG. 6A in a deflected position.

### DETAILED DESCRIPTION

Embodiments of the present disclosure are described in detail with reference to the drawings in which like reference numerals designate identical or corresponding elements in each of the several views. As used herein, the term “distal” refers to that portion of structure farther from the user, while the term “proximal” refers to that portion of structure closer to the user. As used herein, the term “clinician” refers to a doctor, nurse, or other care provider and may include support personnel. In the following description, well-known functions or constructions are not described in detail to avoid obscuring the present disclosure in unnecessary detail.

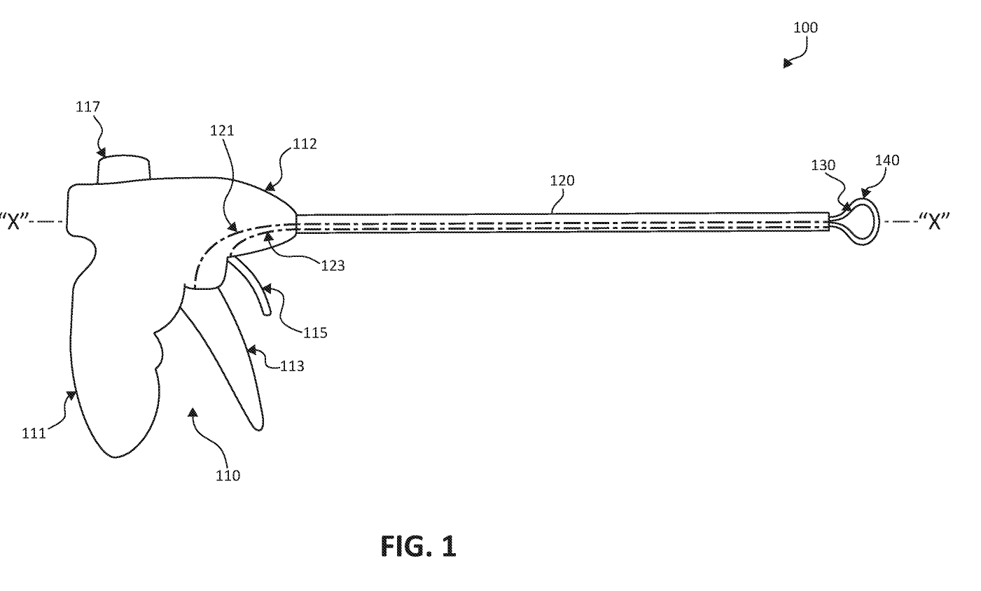

Referring initially to FIG. 1, an electrosurgical instrument in accordance with the present disclosure is shown and designated as 100. Electrosurgical instrument 100 is generally adapted for insertion in a body cavity (e.g., abdominopelvic cavity) through natural orifices, incisions, and/or ports (e.g., vaginal, abdominal, etc.) to resect tissue (e.g., uterine fibroids, cancerous cells, etc.) therein, although electrosurgical instrument 100 may also be used in open surgical procedures. Electrosurgical instrument 100 defines a longitudinal axis “X-X” and generally includes a handle assembly 110, a shaft 120 extending distally from handle assembly 110, a electrosurgical loop 130 operably supported at a distal end portion of shaft 120, and a support structure 140 operably supported at the distal end portion of shaft 120 and disposed about the electrosurgical loop 130.

Handle assembly 110 is configured for gripping and/or using electro surgical instrument 100 and generally includes a fixed handle 111 depending from a housing 112, a first squeezable trigger 113 pivotably attached to fixed handle 111, and a second squeezable trigger 115 pivotably attached to housing 112. Handle assembly 110 may further include a switch 117. First trigger 113 is operatively connected to electrosurgical loop 130 to move electrosurgical loop 130, and second trigger 115 is operatively connected to support structure 140 to move support structure 140, as will be described below, although a single trigger, e.g., first trigger 113, may alternatively be configured to move both electrosurgical loop 130 and support structure 140. Handle assembly 110 may further include a switch 117 that is selectively activatable to energize electrosurgical loop 130. It should be appreciated that handle assembly 110 may include alternative configurations to move one of electrosurgical loop 130 and support structure 140, such as the handle assemblies of commonly owned U.S. Pat. Nos. 8,734,464 and 9,370,341, the entire contents of each of which is hereby incorporated by reference.

Shaft 120 extends distally from handle assembly 110 and is generally an elongated tube configured for insertion into a body cavity, e.g., an abdominopelvic cavity. Shaft 120 includes first and second cables or drive assemblies 121, 123 disposed therein. First drive assembly 121 is connected to first trigger 113 at a proximal end portion thereof and to electrosurgical loop 130 at a distal end portion thereof. Second drive assembly 123 is connected to second trigger 115 at a proximal end portion thereof and to support structure 140 at a distal end portion thereof. Pivoting of first trigger 113 relative to fixed handle 111 thus moves first drive assembly 121 through shaft 120 to move electrosurgical loop 130, and movement of second trigger 115 relative to housing 112 moves second drive assembly 123 to move support structure 140.

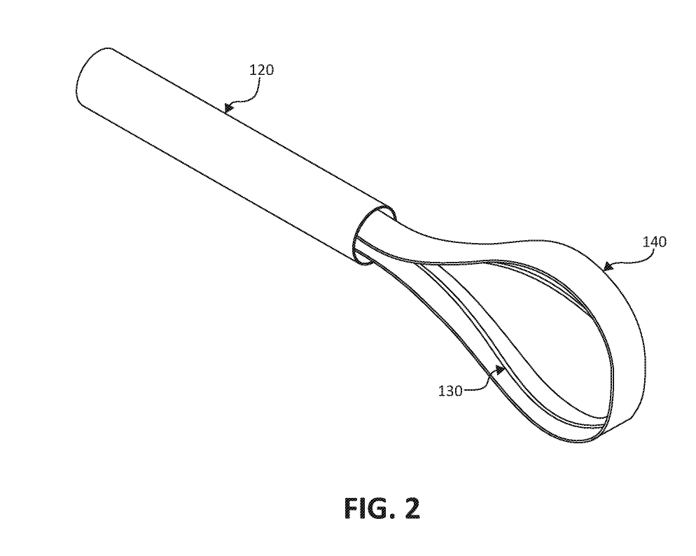

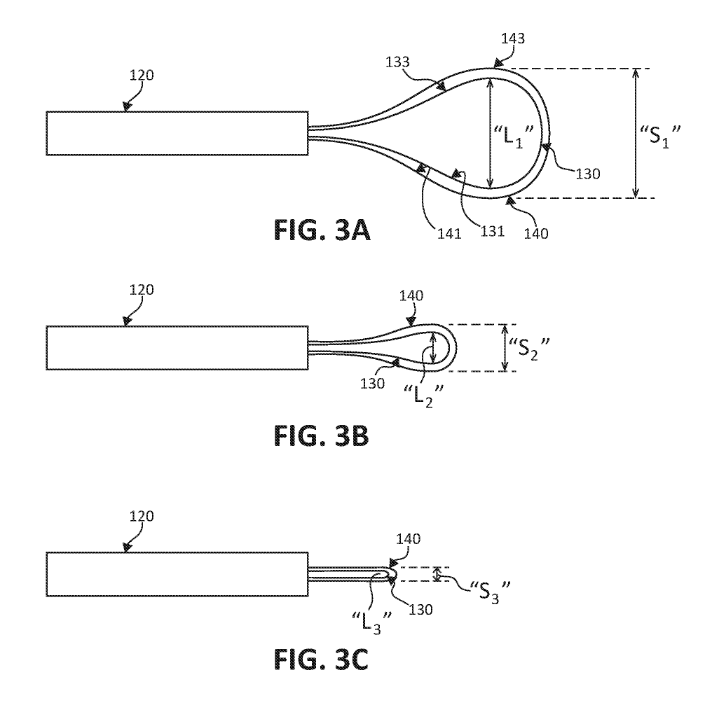

With additional reference to FIGS. 2 and 3A-3C, electrosurgical loop 130 is operatively supported at a distal end portion of shaft 120 and is selectively connected to a source of electrical energy (not shown) and to switch 117 to enable selective energization of electrosurgical loop 130 for resecting tissue therewith. Electrosurgical loop 130 includes an inner peripheral surface 131 and an outer peripheral surface 133. Inner peripheral surface 131 of electrosurgical loop 130 is configured for encircling tissue (e.g., snaring, grasping) to resect tissue at a target site. Outer peripheral surface 133 of electrosurgical loop 130 is configured for resecting tissue and may also be used to move or displace tissue. Movement of first trigger 113 causes electrosurgical loop 130 to move between an expanded state (FIG. 3A) and one or more retracted states (FIGS. 3B and 3C).

With reference to FIGS. 3A, 3B, and 3C, electrosurgical loop 130 defines a diameter “L₁” in the expanded state (FIG. 3A), a diameter “L₂” in the first retracted state (FIG. 3B) that is less than diameter “L₁,” and a diameter “L₃” in the second retracted state (FIG. 3C) that is less than diameter “L₂.” Electrosurgical loop 130 is configured to resect tissue in any and/or all of the expanded, first retracted, and second retracted states. For example, electrosurgical loop 130 can be used to encircle tissue in the expanded state, and then moved to the retracted states to snare tissue to resect tissue. Energization of electrosurgical loop 130 during movement from the expanded state to the retracted states facilitates resection of tissue. Additionally or alternatively, a clinician may move (e.g., rotate, articulate, etc.) handle assembly 120 to provide the clinician with additional control for maneuvering electrosurgical loop 130, when energized, through tissue to resect tissue. Electrosurgical loop 130 may be configured to move (e.g., articulate, rotate, pivot, etc.) about axis “X-X” defined by electrosurgical instrument 100.

Electrosurgical loop 130 may be formed from or include any material having suitable electrical conductivity. For example, electrosurgical loop 130 may be formed from metal, such as, e.g., copper, copper alloy, stainless steel, tungsten, platinum, niobium, molybdenum, etc. Electrosurgical loop 130 may be coated with a conductive material, such as an epoxy coating, fluoropolymer, or the like, to improve the concentration of an electrical field around electrosurgical loop 130 and/or to provide an enhanced electrosurgical effect during a surgical procedure. Electrosurgical loop 130 may be formed as a wire having any suitable cross-sectional configuration, e.g., circular, oval, polygonal, etc.

With continued reference to FIGS. 3A, 3B, and 3C, support structure 140 is operatively supported at a distal end portion of shaft 120 and is disposed about electrosurgical loop 130. Support structure 140 is configured to provide structural reinforcement to electrosurgical loop 130, e.g., for manipulation and resecting of tissue such that electrosurgical loop 130 can be moved or maneuvered without buckling. Support structure 140 is also configured to shield or insulate external surfaces (e.g., specimen bags, healthy tissue, etc.) from exposure to heat or energy from electrosurgical loop 130 when placed in contact or in near relation with such external structures.

Support structure 140 generally defines an inner peripheral surface 141 and an outer peripheral surface 143. Inner peripheral surface 141 of support structure 140 is configured for contact with outer peripheral surface 133 of electrosurgical loop 130, although outer peripheral surface 133 of electrosurgical loop 130 may separate from inner peripheral surface 141 of support structure 140, e.g., where electrosurgical loop 130 is retracted while support structure 140 is maintained in position. Tissue may also be grasped between outer peripheral surface 133 of electrosurgical loop 130 and inner peripheral surface 141 of support structure 140 to provide greater control in maneuvering tissue and resecting tissue. Outer surface 143 of support structure 140 is configured for contact with external surfaces, e.g., healthy tissue, specimen bags, or the like, to shield external structures from heat or energy from electrosurgical loop 130. Support structure 140 generally envelopes or surrounds outer peripheral surface 133 of electrosurgical loop 130.

Movement of second trigger 115 causes support structure 140 to move between an expanded state (FIG. 3A) and first and second retracted states (FIGS. 3B and 3C) about electrosurgical loop 130. Support structure 140 defines a diameter “S₁” in the expanded state (FIG. 3A), a diameter “S₂” in the first retracted state (FIG. 3B) that is less than diameter “S₁,” and a diameter “S₃” in the second retracted state (FIG. 3C) than is less than diameter “S₂.” Support structure 140 may be configured to move (e.g., articulate, rotate, pivot, etc.) about axis “X-X” defined by electrosurgical instrument 100. Support structure 140 may define a band-like configuration having a width larger than that of electrosurgical loop 130 such that support structure 140 completely envelopes or surrounds electrosurgical loop 130. In such embodiments, the band forming support structure 140 may define a flat, concave, convex, chevron, or other suitable cross-sectional configuration. Support structure 140 may be formed from or include any suitable insulative material, such as fiberglass, polystyrene, polyurethane, or the like.

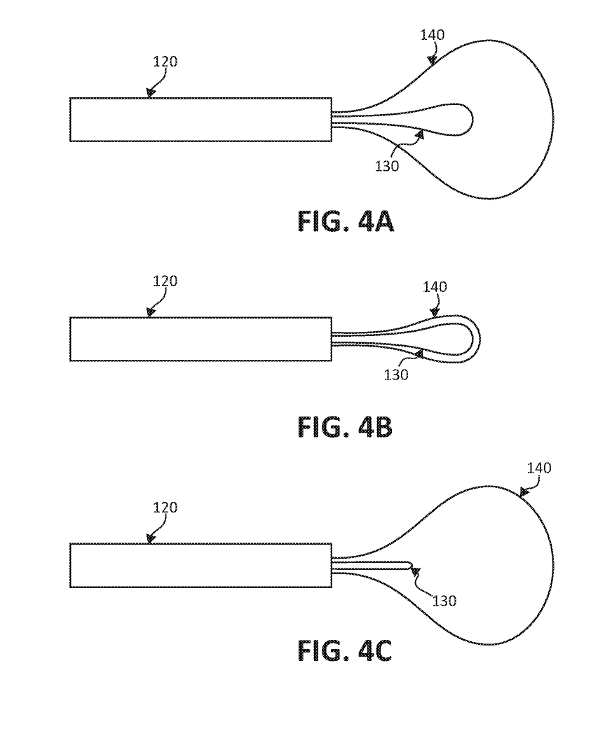

Electrosurgical loop 130 and support structure 140 are movable together with each other, as illustrated in FIGS. 3A-3C, or relative to each other. For example, with reference to FIG. 4A, electrosurgical loop 130 is moved to the first retracted state and support structure 140 is moved to (or maintained in) the extended state. With reference to FIG. 4B, electrosurgical loop 130 has maintained position in the first retracted state and support structure 140 has moved relative to electrosurgical loop 130 from the expanded state into the first retracted state. With reference to FIG. 4C, electrosurgical loop 130 has moved relative to support structure 140 into the second retracted state, while support structure 140 has moved relative to electrosurgical loop 130 back into the expanded state.

It should be appreciated that electrosurgical loop 130 and support structure 140 may be moved into any desired position, and are not limited to those described above. For example, electrosurgical loop 130 and support structure 140 may be extended further outwardly than as shown, and/or may be retracted further inwardly than as shown (e.g., completely within shaft 120). Electrosurgical loop 130 may be fixedly attached to at least a portion of support structure 140 such that e.g., movement of electrosurgical loop 130 causes movement of support structure 140, and/or movement of support structure 140 causes movement of electrosurgical loop 130. For example, support structure 140 may flex or move inwardly into a “V” shape, which may cause a corresponding flexing or movement of electrosurgical loop 130, e.g., to allow for easier separation of tissue during tissue resection.

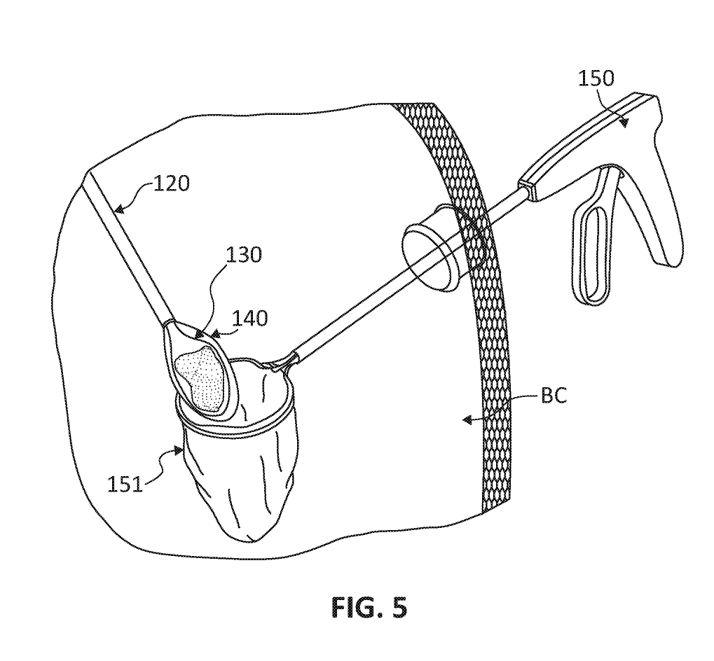

In use, with reference to FIG. 5, shaft 120 of electrosurgical instrument 100 is inserted into a body cavity “BC,” for example, an abdominopelvic cavity. First trigger 113 may be actuated to move electrosurgical loop 130 into a desired position, e.g., expanded state, first retracted state, or second retracted state. Likewise, second trigger 115 may be actuated to move support structure 140 into a desired position relative to electrosurgical loop 130, e.g., expanded state, first retracted state, or second retracted state. Switch 117 is actuated to selectively energize electrosurgical loop 130 with electrical energy such that electrosurgical loop 130 can be used to resect target tissue. First and second triggers 113, 115 may be used to adjust the relative positions of electrosurgical loop 130 and support structure 140, respectively, during the procedure, e.g., to maximize control of the electrosurgical loop 130 during tissue resection without sacrificing performance of electrosurgical loop 130, to provide structural reinforcement to electrosurgical loop 130, and/or to protect external structures from heat and energy from electrosurgical loop 130.

A specimen retrieval device 150 having a specimen bag 151 may be disposed within the body cavity “BC” to enable electrosurgical loop 130 to resect tissue disposed therein. For example, the specimen bag 151 may completely surround the target tissue, and electrosurgical loop 130 may resect the tissue (e.g., morcellate the tissue to reduce tissue size) within specimen bag 151 such that the resected tissue is fully isolated from the body cavity. Alternatively, previously resected tissue may be placed into specimen bag 151 such that the resected tissue is kept separate from healthy tissue. As support structure 140 surrounds electrosurgical loop 130, support structure 140 prevents exposure of specimen bag 151 to heat or energy from electrosurgical loop 130 when resecting tissue therein and/or placing resected tissue therein during a procedure. It is contemplated that electrosurgical loop 130 and support structure 140 may be integrated or attached to inside of specimen bag 151, e.g., such as the mouth or opening of specimen bag 151. In this regard, as tissue is pulled through the mouth of specimen bag 151 (e.g., with a grasper), electrosurgical loop 130 resects the tissue while support structure 140 prevents exposure of specimen bag 151 to heat or energy from electrosurgical loop 130. After completion of the procedure, electrosurgical instrument 100 and specimen retrieval device 150 may be removed from the body cavity “BC.”

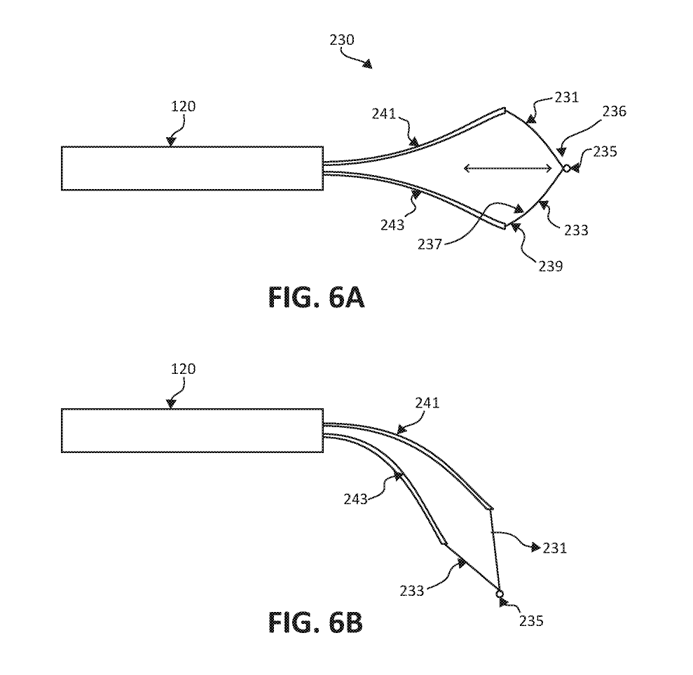

With reference to FIG. 6A and 6B, in accordance with other embodiments of the present disclosure, an electrosurgical loop is shown and generally identified by reference numeral 230. Electrosurgical loop 230 is configured for use with electrosurgical instrument 100 (FIG. 1) and is similar to electrosurgical loop 130 described above, and, thus, will only be described as necessary to demonstrate apparent differences.

Electrosurgical loop 230 is operatively supported at a distal end portion of shaft 120 and is selectively connected to a source of electrical energy (not shown) to energize electrosurgical loop 230 for resecting tissue therewith. Electrosurgical loop 230 includes a first arm 231 and a second arm 233 that is pivotably connected to first arm 231 via a pivot joint 235 at a distal end portion of electrosurgical loop 230.

Pivot joint 235 may include a pivot pin 236 to pivotably connect first and second arms 231, 233. Pivot pin 236 may be formed from an electrically conductive material, e.g., metal, such that electrical energy can flow between first and second arms 231, 233. Alternatively, first and second arms may be integrally and/or monolithically formed (e.g., from a single piece of wire) wherein pivot joint 235 connects first and second arms 231, 233 and includes a coiled, bent, or curved portion that acts as a living hinge to permit first and second arms 231, 233 to move relative to each other.

First and second arms 231, 233 define an inner peripheral surface 237 and an outer peripheral surface 239. Inner peripheral surface 237 of electrosurgical loop 230 is configured for encircling tissue (e.g., snaring or grasping tissue) to resect tissue at a target site. Outer surface 239 of electrosurgical loop 230 is configured for resecting tissue and may be used to move or displace tissue. Electrosurgical instrument 100 (FIG. 1) is selective actuatable to move electrosurgical loop 230 between expanded and retracted states and to energize electrosurgical loop 230, similar to that as described above with respect to electrosurgical loop 130 (FIGS. 3A-3C).

A first stiffening member 241 may be disposed on at least a portion of first arm 231. Likewise, a second stiffening member 243 may be disposed on at least a portion of second arm 233. First and second stiffening members 241, 243 of respective first and second arms 231, 233 are configured to provide structural reinforcement to electrosurgical loop 230 to aid electrosurgical loop 230 in maneuvering through tissue for resecting tissue, and for moving between a rest or non-deflected position (FIG. 6A), wherein electrosurgical loop 230 defines a generally planar configuration, and a deflected position (FIG. 6B), wherein electrosurgical loop 230 defines an arcuate configuration.

First and second stiffening members 241, 243 may be formed from a pliable, shape-memory material that returns electrosurgical loop 230 to the rest or non-deflected position from the deflected position. The shape-memory material may be spring steel, an alloy such as copper-aluminum-nickel or nickel-titanium, or a combination of at least two of zinc, copper, gold, or iron. First and second stiffening members 241, 243 may be conductive or alternatively, non-conductive (e.g. insulative). Other suitable materials and/or configurations of stiffening members 241, 243 are also contemplated.

Electrosurgical loop 230 may be used with support structure 140 (FIG. 2) and may be movable relative to support structure 140, similar to that as described above with respect to electrosurgical loop 130 (FIG. 2).

In use, with additional reference to FIG. 1, actuation of first trigger 113 causes electrosurgical loop 230 to move into a desired position, e.g., an expanded state, first retracted state, second retracted state, etc. Depression of switch 117 energizes electrosurgical loop 230. As the energized electrosurgical loop 230 moves through tissue, first and second stiffening members 241, 243 of respective first and second arms 231, 233 provide structural reinforcement to electrosurgical loop 230 to aid electrosurgical loop 230 in maneuvering through tissue and resecting tissue. However, the exposed portions of first and second arms 231, 233 are permitted to deflect, either by movement into retracted states via actuation of first trigger 113, or through contact with tissue. This deflection, together with first and second arms 231, 233 pivoting towards each other, enables electrosurgical loop 230 to resect tissue into, e.g., substantially cylindrical sections. The resection may be performed within specimen bag 151 of specimen retrieval device 150 (FIG. 5) or, alternatively, the previously resected tissue may be placed in specimen bag 151 of specimen retrieval device 150 (FIG. 5). The clinician can reset electrosurgical loop 230 to the extended position by movement of first trigger 113, whereupon stiffening members 241, 243 will cause electrosurgical loop 230 to move to the rest or non-deflected position to enable further ensnaring and resecting of tissue, as needed.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. An electrosurgical instrument, comprising:
a handle assembly including an actuator;
a shaft extending distally from the handle assembly;
an electrosurgical loop adapted to connect to a source of energy and operably supported at a distal end portion of the shaft, the electrosurgical loop movable relative to the shaft between a first retracted state and a first expanded state;
a support structure operably supported at the distal end portion of the shaft and disposed about the electrosurgical loop, the support structure movable relative to the shaft between a second retracted state and a second expanded state; and
wherein actuation of the actuator moves at least one of the electrosurgical loop or the support structure from the retracted state to the expanded state, the electrosurgical loop and the support structure movable relative to each other.

2. The electrosurgical instrument according to claim 1, wherein the support structure includes an inner surface and an outer surface, the inner surface configured for contact with the electrosurgical loop and the outer surface configured for contact with an external surface, the support structure configured to shield the external surface from heat and energy from the electrosurgical loop.

3. The electrosurgical instrument according to claim 1, wherein the support structure is a band disposed about an outer periphery of the electrosurgical loop, the band defining a width that is greater than a width of the electrosurgical loop.

4. The electrosurgical instrument according to claim 1, wherein the electrosurgical loop includes a metal selected from the group consisting of copper, copper alloy, stainless steel, tungsten, platinum, niobium, and molybdenum.

5. The electrosurgical instrument according to claim 1, wherein the actuator is a movable trigger movable relative to a fixed handle of the handle assembly.

6. The electrosurgical instrument according to claim 1, further comprising a second actuator coupled to the handle assembly, the actuator configured to move the electrosurgical loop relative to the shaft and the second actuator configured to move the support structure relative to the shaft.

7. The electrosurgical instrument according to claim 1, wherein the electrosurgical loop is connected to a source of electrical energy to energize the electrosurgical loop for resection of tissue therewith.

8. The electrosurgical instrument according to claim 1, wherein the electrosurgical loop includes a sleeve surrounding a portion of the electrosurgical loop.

9. The electrosurgical instrument according to claim 1, wherein the support structure is configured to provide structural reinforcement to the electrosurgical loop.

10. The electrosurgical instrument according to claim 1, wherein the support structure includes an insulative material.

11. An electrosurgical instrument, comprising:
a handle assembly including at least one actuator;
a shaft extending distally from the handle assembly;
an electrosurgical loop operably supported at a distal end portion of the shaft, the electrosurgical loop including first and second arms pivotably connected to each other at distal end portions thereof, the first and second arms pivotable relative to one another to move the electrosurgical loop between a retracted state and an expanded state; and
a first stiffening sleeve disposed partially about the first arm and a second stiffening sleeve disposed partially about the second arm, the first and second stiffening sleeves configured to provide structural reinforcement to the electrosurgical loop.

12. The electrosurgical instrument according to claim 11, wherein a pivot pin pivotably connects the first and second arms to each other.

13. The electrosurgical instrument according to claim 12, wherein the pivot pin is formed from an electrically conductive material configured to permit electrical energy transmission between the first and second arms.

14. The electrosurgical instrument according to claim 11, wherein the first and second stiffening sleeves are formed from a spring steel configured to enable the electrosurgical loop to move between a non-deflected position and a deflected position.

15. The electrosurgical instrument according to claim 11, further comprising a band disposed about an outer periphery of the electrosurgical loop, the band defining a width that is greater than a width of the electrosurgical loop.

16. The electrosurgical instrument according to claim 15, wherein the band includes an inner surface and an outer surface, the inner surface configured for contact with the electrosurgical loop and the outer surface configured for contact with an external surface, the band configured to shield the external surface from heat and energy from the electrosurgical loop.

17. The electrosurgical instrument according to claim 16, wherein the at least one actuator includes a first trigger and a second trigger, the first trigger configured to move the electrosurgical loop and the second actuator configured to move the band about the electrosurgical loop, the electrosurgical loop and the band movable relative to each other.

18. The electrosurgical instrument according to claim 11, wherein the electrosurgical loop is connected to a source of electrical energy to energize the electrosurgical loop for resection of tissue therewith.

19. The electrosurgical instrument according to claim 11, wherein the electrosurgical loop includes a metal selected from the group consisting of copper, copper alloy, stainless steel, tungsten, platinum, niobium, and molybdenum.
